(19) United States
(12) Patent Application Publication
(10) Pub. No.: US 2025/0057537 A1
(43) Pub. Date: Feb. 20, 2025

(54) DELIVERY DEVICE FOR OCCLUSIVE IMPLANTS

(71) Applicant: Boston Scientific Scimed, Inc., Maple Grove, MN (US)

(72) Inventors: Carly Rogers, Eden Prairie, MN (US); Sara Reda Eskander, Plymouth, MN (US); Daniel James Viola, Minneapolis, MN (US); Mitchell Nelson, Hudson, WI (US); David John Onushko, Maple Grove, MN (US)

(73) Assignee: Boston Scientific Scimed, Inc., Maple Grove, MN (US)

(21) Appl. No.: 18/805,774

(22) Filed: Aug. 15, 2024

Related U.S. Application Data
(60) Provisional application No. 63/533,025, filed on Aug. 16, 2023.

(51) Int. Cl. A61B 17/12; A61B 17/00

(52) U.S. Cl. CPC A61B 17/12122; A61B 17/00234; A61B 17/12031; A61B 17/12172; A61B 2017/00243; A61B 2017/00305; A61B 2017/00477; A61B 2017/12054

(57) ABSTRACT

Delivery devices for an occlusive implant and method for making and using delivery devices are disclosed. An example delivery device may include a delivery sheath having a proximal region and a laser cut distal tip region. A reinforcing member may extend along the proximal region. The laser cut distal tip region may have one or more laser cuts formed therein that form a patterned distal section. The patterned distal section may be configured to shift between a delivery configuration and an expanded configuration. A core member may be slidably disposed within the delivery sheath. An occlusive implant may be releasably coupled to the core member.

### CROSS-REFERENCE TO RELATED APPLICATIONS

This application claims the benefit of U.S. Provisional Patent Application Ser. No. 63/533,025, filed Aug. 16, 2023, which is incorporated herein by reference.

### TECHNICAL FIELD

The present disclosure pertains to medical devices, and methods for manufacturing medical devices. More particularly, the present disclosure pertains to delivery devices for occlusive implants.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A delivery device for an occlusive implant is disclosed. The delivery device comprises: a delivery sheath having a proximal region and a laser cut distal tip region; a reinforcing member extending along the proximal region; wherein the laser cut distal tip region has one or more laser cuts formed therein that form a patterned distal section, the patterned distal section being configured to shift between a delivery configuration and an expanded configuration; a core member slidably disposed within the delivery sheath; and an occlusive implant releasably coupled to the core member.

Alternatively or additionally to any of the embodiments above, at least some of the one or more laser cuts go completely through a wall surface of the laser cut distal tip region.

Alternatively or additionally to any of the embodiments above, at least some of the one or more laser cuts go only partially through a wall surface of the laser cut distal tip region.

Alternatively or additionally to any of the embodiments above, the one or more laser cuts all extend the same distance proximally along the laser cut distal tip region.

Alternatively or additionally to any of the embodiments above, at least some of the one or more laser cuts extend a different distance proximally along the laser cut distal tip region.

Alternatively or additionally to any of the embodiments above, the patterned distal section includes a plurality of rounded projections.

Alternatively or additionally to any of the embodiments above, the patterned distal section includes a plurality of pointed projections.

Alternatively or additionally to any of the embodiments above, the patterned distal section includes axially-extending grooves.

Alternatively or additionally to any of the embodiments above, the patterned distal section includes circumferentially-extending grooves.

Alternatively or additionally to any of the embodiments above, the patterned distal section includes overlapping grooves.

A delivery device for an occlusive implant is disclosed. The delivery device comprises: a delivery sheath having a proximal region and a distal tip region; wherein the distal tip region includes a patterned distal section; wherein the patterned distal section is configured to allow the distal tip region to shift between a delivery configuration and an expanded configuration; and a proximal reinforcing member disposed along the proximal region.

Alternatively or additionally to any of the embodiments above, the patterned distal section includes laser cuts that go completely through a wall surface of the distal tip region.

Alternatively or additionally to any of the embodiments above, the patterned distal section includes laser cuts that go only partially through a wall surface of the distal tip region.

Alternatively or additionally to any of the embodiments above, the patterned distal section includes laser cuts that all extend the same distance proximally along the distal tip region.

Alternatively or additionally to any of the embodiments above, the patterned distal section includes laser cuts that extend a differing distance proximally along the distal tip region.

Alternatively or additionally to any of the embodiments above, the patterned distal section includes a plurality of rounded projections.

Alternatively or additionally to any of the embodiments above, the patterned distal section includes a plurality of pointed projections.

Alternatively or additionally to any of the embodiments above, the patterned distal section includes axially-extending grooves, circumferentially-extending grooves, or both.

Alternatively or additionally to any of the embodiments above, further comprising a core member slidably disposed within the delivery sheath and an occlusive implant releasably coupled to the core member.

A method for implanting an occlusive implant into a left atrial appendage is disclosed. The method comprises: advancing a delivery device to a position adjacent to the left atrial appendage, the delivery device comprising: a delivery sheath having a proximal region and a laser cut distal tip region, a reinforcing member extending along the proximal region, wherein the laser cut distal tip region has one or more laser cuts formed therein that form a patterned distal section, the patterned distal section being configured to shift between a delivery configuration and an expanded configuration, a core member slidably disposed within the delivery sheath, and an occlusive implant releasably coupled to the core member; and advancing the core member such that the occlusive implant advances out from the laser cut distal tip region.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example delivery system.

FIG. 2 is a side view of an example delivery system.

FIG. 3 is a side view of a portion of an example delivery system.

FIG. 4 is a side view of a portion of an example delivery system.

FIG. 5 is a side view of a portion of an example delivery system.

FIG. 6 is a side view of a portion of an example delivery system.

FIG. 7 is a side view of a portion of an example delivery system.

FIG. 8 is a side view of a portion of an example delivery system.

FIG. 9 is a side view of a portion of an example delivery system.

FIG. 10 is a side view of a portion of an example delivery system.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

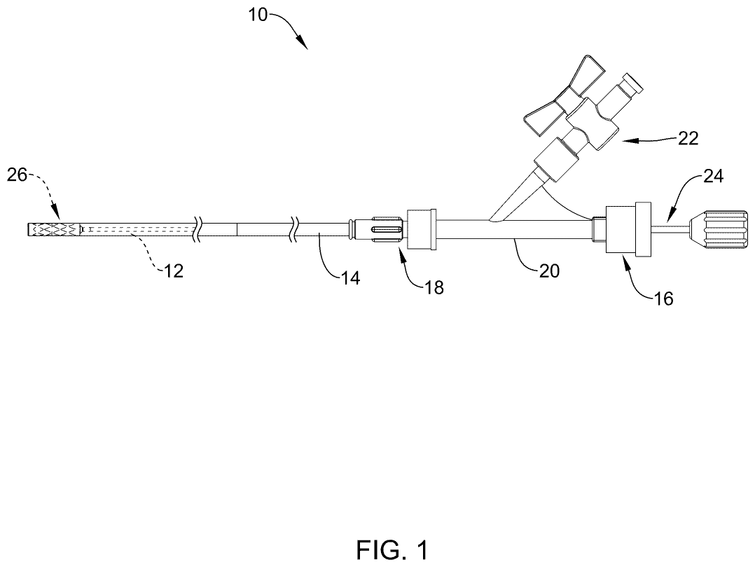

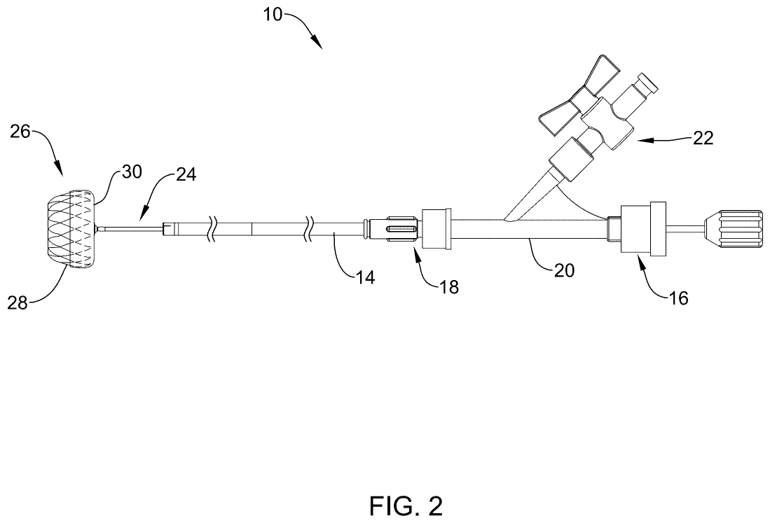

FIGS. 1-2 schematically illustrate selected components and/or arrangements of an occlusive implant system. It should be noted that in any given figure, some features of the occlusive implant system may not be shown, or may be shown schematically, for simplicity. Additional details regarding some of the components of the occlusive implant system may be illustrated in other figures in greater detail. The occlusive implant system may be used to deliver and/or deploy a variety of medical implants (e.g., a cardiovascular implant, an occlusive implant, etc.) to one or more locations within the anatomy, including but not limited to, in some embodiments, the heart and/or the left atrial appendage. In the interest of clarity, the following discussion refers to an occlusive implant, but other medical implants may be used and/or considered with the occlusive implant system.

The occlusive implant system may include a delivery system 10 including a delivery sheath 14 having a delivery lumen 12 extending proximally from a distal end of the delivery sheath 14. In one example, the delivery lumen 12 extends from a proximal opening to a distal opening of the delivery sheath 14. The delivery system 10 may include a proximal hub 16. In some embodiments, the delivery system may include a mid-hub 18. In some embodiments, the delivery system 10 may include a mid-shaft 20 extending from the proximal hub 16 to the mid-hub 18. In some embodiments, the delivery sheath 14 may extend distally from the mid-hub 18. Other configurations are also contemplated. In some embodiments, the delivery system 10 may include a side port 22. In some embodiments, the side port 22 may be in communication with the mid-shaft 20. Other configurations are also contemplated. In some embodiments, the delivery system 10 and/or the delivery lumen 12 may include a proximal segment (not shown) extending within and/or through the mid-hub 18, the mid-shaft 20, and the proximal hub 16. In some embodiments, the proximal segment may be in fluid communication with and/or may be an extension of the delivery lumen 12 of the delivery sheath 14. In some embodiments, the side port 22 may be in fluid communication with the proximal segment and/or the delivery lumen 12.

The occlusive implant system and/or the delivery system 10 may include a core member or core wire 24 slidably and/or rotatably disposed within the delivery lumen 12 (and the proximal segment, where present). The occlusive implant system may include an occlusive implant 26, which may be configured for implantation within a left atrial appendage, releasably engaged with and/or releasably attached to a distal end of the core wire 24. In at least some embodiments, the occlusive implant 26 may be a left atrial appendage closure device. In some embodiments, a proximal end of the core wire 24 may extend proximally of a proximal end of the delivery sheath 14 and/or the proximal opening of the delivery lumen 12 for manual manipulation by a clinician or practitioner. In at least some embodiments, the delivery sheath 14 may comprise and/or may be formed from a polymeric material. In some embodiments, the delivery sheath 14 may comprise and/or may be formed from a plurality of polymeric materials. In some embodiments, the delivery sheath may comprise and/or may be formed from a combination of metallic and polymeric materials. In some embodiments, the delivery sheath 14 may include a reinforcing element, such as a mesh, a coil, a braid, etc., formed therein, embedded therein, attached thereto, etc. along at least a portion of a length of the delivery sheath 14. Other configurations are also contemplated. Some suitable, but non-limiting, examples of materials for the occlusive implant system, the core wire 24, and/or the delivery sheath 14, etc., including but not limited to metallic materials, polymeric materials, etc., are discussed below.

The occlusive implant 26 may include an expandable framework 28 (e.g., FIG. 2) configured to shift between a delivery configuration (e.g., FIG. 1), such as when the occlusive implant 26 is disposed within the delivery lumen 12 proximate the distal opening and/or within a distal portion of the delivery lumen 12, and a deployed configuration (e.g., FIG. 2) when the occlusive implant 26 is unconstrained by the delivery sheath 14.

In some embodiments, the expandable framework 28 may comprise a plurality of interconnected struts. In some embodiments, the expandable framework 28 may be compliant or semi-compliant and may generally conform to and/or be configured to sealingly engage with the shape and/or geometry of the left atrial appendage in the deployed configuration.

In some embodiments, a proximal end of the expandable framework 28 may be configured to releasably attach, join, couple, engage, or otherwise connect to the distal end of the core wire 24 (e.g., FIG. 2). In some embodiments, the proximal end of the expandable framework 28 may include a proximal hub coupled and/or non-releasably attached thereto. In some embodiments, the proximal hub may be configured to and/or adapted to releasably couple with, join to, mate with, or otherwise engage a distal end of the core wire 24. Other means of releasably coupling and/or engaging the expandable framework 28 to the distal end of the core wire 24 are also contemplated.

In some embodiments, the occlusive implant 26 may include an occlusive element 30 (e.g., a membrane, a fabric, or a tissue element, etc.) connected to, disposed on, disposed over, disposed about, or covering at least a portion the expandable framework 28. In some embodiments, the occlusive element 30 may be connected to, disposed on, disposed over, disposed about, or cover at least a portion of an outer (or outwardly facing) surface of the expandable framework 28.

In some embodiments, the occlusive element 30 may be permeable or impermeable to blood and/or other fluids, such as water. In some embodiments, the occlusive element 30 may include a polymeric membrane, a metallic or polymeric mesh, a porous or semi-porous filter-like material, or other suitable construction. In some embodiments, the occlusive element 30 prevents thrombi (e.g., blood clots, etc.) from passing through the occlusive element 30 and out of the left atrial appendage into the blood stream. In some embodiments, the occlusive element 30 promotes endothelization after implantation, thereby effectively removing the target site (e.g., the left atrial appendage, etc.) from the patient's circulatory system. Some suitable, but non-limiting, examples of materials for the occlusive element 30 are discussed below.

In some embodiments, the expandable framework 28 and/or the plurality of interconnected struts may be integrally formed and/or cut from a unitary member. In some embodiments, the expandable framework 28 and/or the plurality of interconnected struts may be integrally formed and/or cut from a unitary tubular member and subsequently formed and/or heat set to a desired shape in the deployed configuration. In some embodiments, the expandable framework 28 and/or the plurality of interconnected struts may be integrally formed and/or cut from a unitary flat member or sheet, and then rolled or formed into a tubular structure and subsequently formed and/or heat set to the desired shape in the deployed configuration. Some exemplary means and/or methods of making and/or forming the expandable framework 28 include laser cutting, machining, punching, stamping, electro discharge machining (EDM), chemical dissolution, etc. Other means and/or methods are also contemplated.

In use, the delivery sheath 14 may be advanced and/or navigated to the left atrial appendage to deliver the occlusive implant 26 thereto. In one example, the delivery sheath 14 may be advanced and/or navigated to the left atrial appendage using and/or over a guidewire. For example, the delivery sheath 14 may be advanced to the patient's left atrium and the distal end disposed adjacent to the left atrial appendage with the occlusive implant 26 disposed therein in the delivery configuration. In some embodiments, the delivery sheath 14 may include steering capability. After the distal end of the delivery sheath 14 is disposed adjacent to and/or at the left atrial appendage, the core wire 24 may be advanced distally relative to the delivery sheath 14 to advance the occlusive implant 26 out of the delivery sheath 14, where the occlusive implant 26 may shift to the deployed configuration.

While not expressly illustrated, in some embodiments, the occlusive implant system may further comprise an access device. In some embodiments, the access device may be a bi-directional steerable catheter and/or an intravascular catheter. Examples of intravascular catheters may include, but are not limited to, balloon catheters, atherectomy catheters, device delivery catheters, drug delivery catheters, diagnostic catheters, and guide catheters.

In some embodiments, the access device may be advanced and/or navigated to the left atrial appendage. In one example, the access device may be advanced and/or navigated to the left atrial appendage using and/or over a guidewire. For example, the access device may be advanced to the patient's left atrium and a distal tip disposed adjacent to the left atrial appendage. In some embodiments, the access device may include steering capability. In some embodiments, the delivery system 10 may be inserted through the access device. In some embodiments, the length of the delivery sheath 14 may be substantially equal to the length of the access device. In some embodiments, the length of the delivery sheath 14 may be slightly longer than the access device. During use, the delivery sheath 14 may be advanced within the access device with the occlusive implant 26 disposed therein in the delivery configuration. After the distal end of the delivery sheath 14 is disposed adjacent to and/or at the distal end of the access device, the core wire 24 may be advanced distally relative to the delivery sheath 14 and/or the access device to advance the occlusive implant 26 out of the delivery sheath 14 and the access device, where the occlusive implant 26 may shift to the deployed configuration.

In some embodiments, the delivery system, the delivery sheath 14, and/or the access device may be sized in accordance with its intended use. For example, the delivery system, the delivery sheath 14, and/or the access device can have a length that is in the range of about 10 to about 150 centimeters, about 25 to about 125 centimeters, about 50 to about 100 centimeters, about 25 centimeters to about 50 centimeters, about 50 to about 75 centimeters, about 75 to about 100 centimeters, etc. Other lengths are also contemplated, including but not limited to subsets of ranges disclosed herein. It is further contemplated that the outer diameter of the delivery system, the delivery sheath 14, and/or the access device may vary based on the use or application. In some examples, the outer diameter of the delivery system, the delivery sheath 14, and/or the access device may be about 2 millimeters (mm), about 3 mm (or 9 French), about 3.5 mm, about 4 mm (or 12 French), about 4.5 mm, about 5 mm (or 15 French), about 5.33 mm, about 5.5 mm, about 5.66 mm (or 17 French), about 6 mm, about 6.5 mm, about 7 mm (or 21 French), about 8 mm, or other suitable sizes. In some embodiments, the outer diameter of the delivery system, the delivery sheath 14, and/or the access device may be a maximum of 5.66 mm (17 French) and is preferably smaller than 5.66 mm (17 French). Other configurations are also contemplated. In some embodiments, it is desirable for the outer diameter of the delivery system, the delivery sheath 14, and/or the access device to be as small as possible.

In some instances, during a procedure, it may be necessary to recapture and/or reposition the occlusive implant 26. For example, the initial placement of the occlusive implant 26 may be incorrect and/or inadequate. Accordingly, the delivery sheath 14 and/or the access device may be configured to permit recapture and/or reposition of the occlusive implant 26. Disclosed herein are delivery systems that include a delivery sheath, for example, with proximal region, a distal tip region, and a patterned distal section. These and/or other structures may help to make the delivery sheath more amenable to recapture and/or repositioning of the occlusive implant. Some additional details regarding these and other structures are disclosed herein.

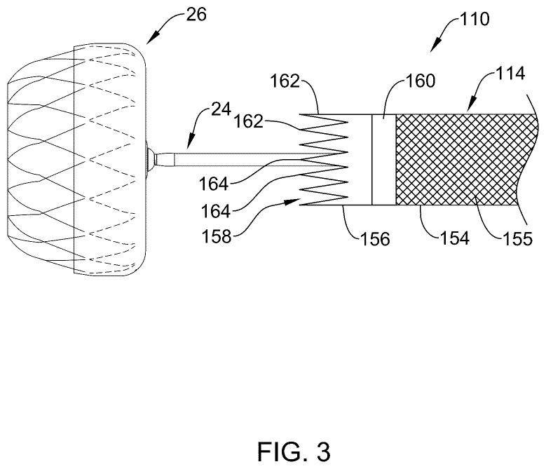

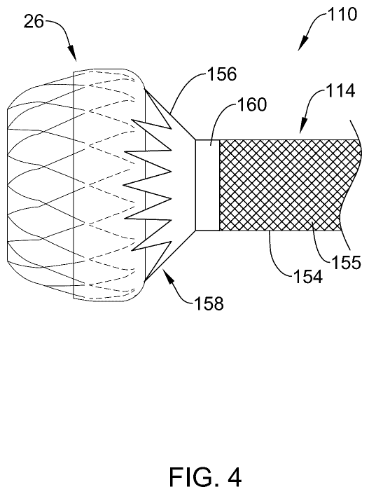

FIGS. 3-4 illustrate a portion of another example delivery device 110 (e.g., for an occlusive implant) that may be similar in form and function to other delivery devices disclosed herein. The delivery device 110 may include a delivery sheath 114 having a proximal region 154 and a distal tip region 156. The proximal region 154 may include a reinforcing member 155. In some instances, the reinforcing member 155 may take the form of a braid. Other reinforcing members are contemplated including coils, meshes, wires, and/or the like. The distal tip region 156 may include a base 160. In some instances, the base 160 may include or otherwise define a radiopaque marker or band that may help to visualize the distal tip region 156 during an intervention.

The distal tip region 156 may include a patterned distal section 158. In general, the patterned distal section 158 (and/or other patterned distal sections disclosed herein) are configured to allow the distal tip region 156 (and/or the patterned distal section 158) to shift between a first or delivery configuration and a second or expanded configuration. The expanded configuration may help an occlusive implant (e.g., such as the occlusive implant 26) to be efficiently guided into the distal tip region 156, for example during a recapture and/or repositioning process.

The patterned distal section 158 may include a plurality of distal arm segments 162 that, for example, may generally extend from the base 160. In general, the distal arm segments 162 may be configured to shift between a first or delivery configuration (e.g., as shown in FIG. 3) and a second or expanded configuration (e.g., as shown in FIG. 4). When in the expanded configuration, the patterned distal section 158 may help to hold or maintain the distal tip region 156 into a shape that resembles a funnel and/or otherwise provide a funnel-like shape to the distal tip region 156 (e.g., as shown in FIG. 4). This may help to guide the occlusive implant 26 into the distal tip region 156 (e.g., by actuating the core wire 24), for example when it is desirable to recapture and/or reposition the implant. The funnel-like shape may help to reduce damage to the delivery sheath 114 during a recapture and/or repositioning process.

In at least some instances, the distal arm segments 162 (and/or the patterned distal section 158) may be formed by laser cutting the distal tip region 156. Laser cutting may be desirable for a number of reasons. For example, laser cutting the distal tip region 156 may allow for relatively smooth, clean cuts to be formed in a variety of patterns/shapes. In FIGS. 3-4, the pattern of the patterned distal section 158 can be described as having arms (e.g., distal arm segments 162) or projections with pointed ends 164. In some instances, the pattern may be described as star-shaped. A variety of other patterns/shapes are contemplated.

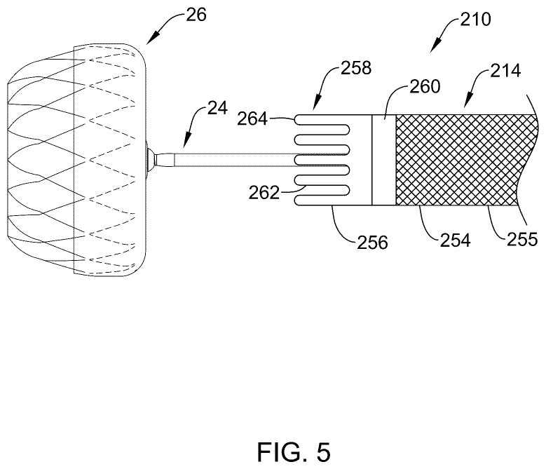

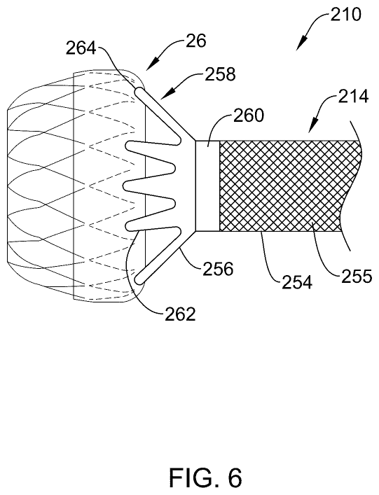

FIGS. 5-6 illustrate a portion of another example delivery device 210 (e.g., for an occlusive implant) that may be similar in form and function to other delivery devices disclosed herein. The delivery device 210 may include a delivery sheath 214 having a proximal region 254 and a distal tip region 256. The proximal region 254 may include a reinforcing member 255. In some instances, the reinforcing member 255 may take the form of a braid. Other reinforcing members are contemplated including coils, meshes, wires, and/or the like. The distal tip region 256 may include a patterned distal section 258. In general, the patterned distal section 258 (and/or other patterned distal sections disclosed herein) are configured to allow the distal tip region 256 (and/or the patterned distal section 258) to shift between a first or delivery configuration and a second or expanded configuration. The expanded configuration may help an occlusive implant (e.g., such as the occlusive implant 26) to efficiently be guided into the distal tip region 256, for example during a recapture and/or repositioning process.

The distal tip region 256 may include a base 260. In some instances, the base 160 may include or otherwise define a radiopaque marker or band that may help to visualize the distal tip region 256 during an intervention. The patterned distal section 258 may include a plurality of distal arm segments 262 that, for example, may extend from the base 260. In general, the distal arm segments 262 may be configured to shift between a first configuration (e.g., as shown in FIG. 5) and an expanded configuration (e.g., as shown in FIG. 6). When in the expanded configuration, the patterned distal section 258 may help to hold or maintain the distal tip region 256 into a shape that resembles a funnel and/or otherwise provide a funnel-like shape to the distal tip region 256 (e.g., as shown in FIG. 2). This may help to guide the occlusive implant 26 into the distal tip region 256 (e.g., by actuating the core wire 24), for example when it is desirable to recapture and/or reposition the implant. The funnel-like shape may help to reduce damage to the delivery sheath 214 during a recapture and/or repositioning process.

In at least some instances, the distal arm segments 262 (and/or the patterned distal section 158) may be formed by laser cutting the distal tip region 156. Laser cutting may be desirable for a number of reasons. For example, laser cutting the distal tip region 256 may allow for relatively smooth, clean cuts to be formed in a variety of patterns/shapes. In FIGS. 5-6, the pattern of the patterned distal section 258 can be described as having arms (e.g., distal arm segments 262) or projections with rounded ends 264. In some instances, the pattern may be described as petal-shaped. A variety of other patterns/shapes are contemplated.

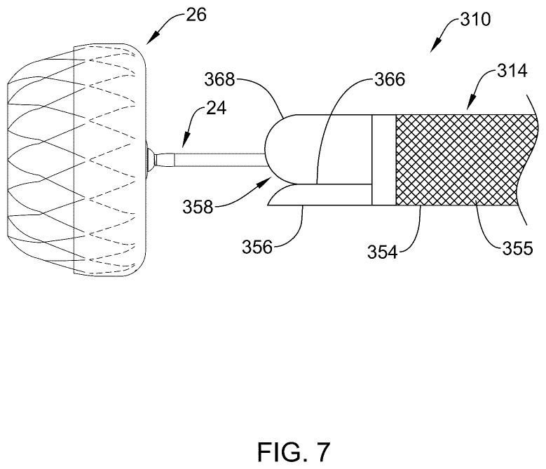

FIG. 7 illustrates a portion of another example delivery device 310 (e.g., for an occlusive implant) that may be similar in form and function to other delivery devices disclosed herein. The delivery device 310 may include a delivery sheath 314 having a proximal region 354 and a distal tip region 356. The proximal region 354 may include a reinforcing member 355. In some instances, the reinforcing member 355 may take the form of a braid. Other reinforcing members are contemplated including coils, meshes, wires, and/or the like. The distal tip region 356 may include a patterned distal section 358. In general, the patterned distal section 358 (and/or other patterned distal sections disclosed herein) are configured to allow the distal tip region 356 (and/or the patterned distal section 358) to shift between a first or delivery configuration and a second or expanded configuration. The expanded configuration may help an occlusive implant (e.g., such as the occlusive implant 26) to efficiently be guided into the distal tip region 356, for example during a recapture and/or repositioning process.

In at least some instances, the patterned distal section 358 may be formed by laser cutting the distal tip region 356. Laser cutting may be desirable for a number of reasons. For example, laser cutting the distal tip region 356 may allow for relatively smooth, clean cuts to be formed in a variety of patterns/shapes. In the example shown in FIG. 7, the patterned distal section 358 may be characterized by axial slits 366 and rounded end portions 368.

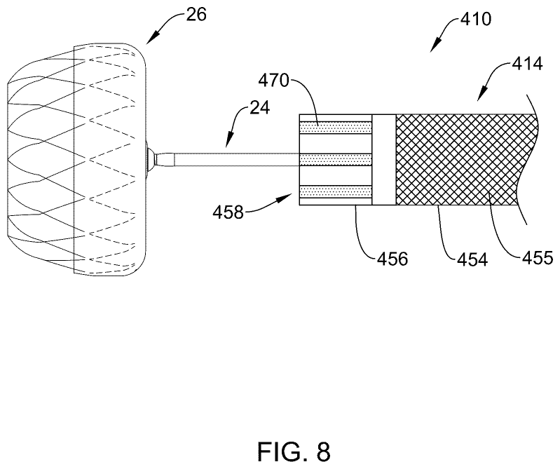

The patterned distal sections 158, 258, 358 depicted in FIGS. 3-7 may include cuts (e.g. laser cuts) that extend all the way through the wall of the delivery sheaths 114, 214, 314 (e.g., distal tip regions 156, 256, 356). Other arrangements are contemplated including arrangements where the pattered distal end region is formed by partially cutting or ablating only part of the way through the wall of the delivery sheaths. For example, FIG. 8 illustrates a portion of another example delivery device 410 (e.g., for an occlusive implant) that may be similar in form and function to other delivery devices disclosed herein. The delivery device 410 may include a delivery sheath 414 having a proximal region 454 and a distal tip region 456. The proximal region 454 may include a reinforcing member 455.

The distal tip region 456 may include a patterned distal section 458. In general, the patterned distal section 458 (and/or other patterned distal sections disclosed herein) are configured to allow the distal tip region 456 (and/or the patterned distal section 458) to shift between a first or delivery configuration and a second or expanded configuration. The expanded configuration may help an occlusive implant (e.g., such as the occlusive implant 26) to efficiently be guided into the distal tip region 456, for example during a recapture and/or repositioning process. In some instances, the patterned distal section 458 may be formed by ablating the distal tip region 456. In the example shown in FIG. 8, the patterned distal section 458 may be characterized by axial grooves 466. The process used to ablate the distal tip region 456 may vary. In some instances, a laser may be used (e.g., laser cutting and/or laser ablating). Other processes are contemplated including mechanically ablating, chemically ablating, thermally ablating, combinations thereof, and/or the like.

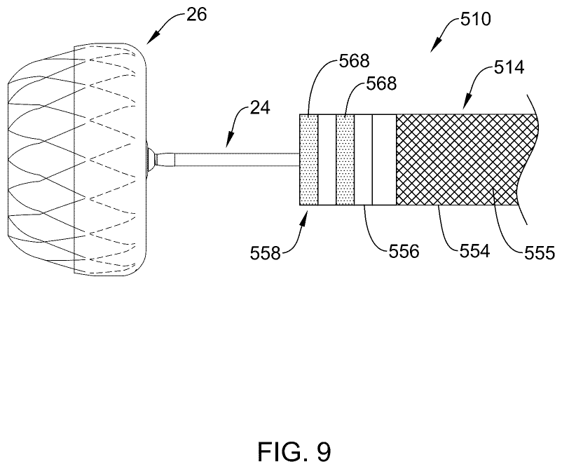

FIG. 9 illustrates a portion of another example delivery device 510 (e.g., for an occlusive implant) that may be similar in form and function to other delivery devices disclosed herein. The delivery device 510 may include a delivery sheath 514 having a proximal region 554 and a distal tip region 556. The proximal region 554 may include a reinforcing member 555.

The distal tip region 556 may include a patterned distal section 558. In general, the patterned distal section 558 (and/or other patterned distal sections disclosed herein) are configured to allow the distal tip region 556 (and/or the patterned distal section 558) to shift between a first or delivery configuration and a second or expanded configuration. The expanded configuration may help an occlusive implant (e.g., such as the occlusive implant 26) to efficiently be guided into the distal tip region 556, for example during a recapture and/or repositioning process. In some instances, the patterned distal section 558 may be formed by ablating the distal tip region 556. In the example shown in FIG. 9, the patterned distal section 558 may be characterized by circumferential grooves 568. The process used to ablate the distal tip region 556 may vary. In some instances, a laser may be used (e.g., laser cutting and/or laser ablating). Other processes are contemplated including mechanically ablating, chemically ablating, thermally ablating, combinations thereof, and/or the like.

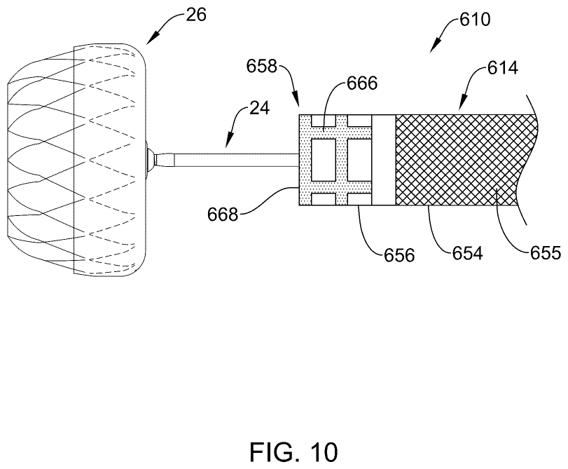

FIG. 10 illustrates a portion of another example delivery device 610 (e.g., for an occlusive implant) that may be similar in form and function to other delivery devices disclosed herein. The delivery device 610 may include a delivery sheath 614 having a proximal region 654 and a distal tip region 656. The proximal region 654 may include a reinforcing member 655.

The distal tip region 656 may include a patterned distal section 658. In general, the patterned distal section 658 (and/or other patterned distal sections disclosed herein) are configured to allow the distal tip region 656 (and/or the patterned distal section 658) to shift between a first or delivery configuration and a second or expanded configuration. The expanded configuration may help an occlusive implant (e.g., such as the occlusive implant 26) to efficiently be guided into the distal tip region 656, for example during a recapture and/or repositioning process. In some instances, the patterned distal section 658 may be formed by ablating the distal tip region 656. In the example shown in FIG. 10, the patterned distal section 658 may be characterized by axial grooves 666 and circumferential grooves 668. For example, the axial grooves 666 and circumferential grooves 668 may overlap or otherwise be arranged in an overlapping arrangement. The process used to ablate the distal tip region 656 may vary. In some instances, a laser may be used (e.g., laser cutting and/or laser ablating). Other processes are contemplated including mechanically ablating, chemically ablating, thermally ablating, combinations thereof, and/or the like.

In some instances, the distal tip regions 156, 256, 356, 456, 656 disclosed herein may include one or more slits or cutouts. The slits may form or otherwise define arm segments or flaps. The flaps may be configured to aid in expansion of the distal tip regions 156, 256, 356, 456, 656. In other words, the flaps may allow the distal tip regions 156, 256, 356, 456, 656 to open up or expand into an enlarged shape/configuration (e.g., which may resemble a funnel). Any of the distal tip regions 156, 256, 356, 456, 656 may include slits and flaps, as appropriate, even if not expressly illustrated.

The materials that can be used for the various components of the delivery system 10 (and/or other delivery systems disclosed herein) may include those commonly associated with medical devices. For simplicity purposes, the following discussion makes reference to the delivery system 10. However, this is not intended to limit the devices and methods described herein, as the discussion may be applied to other delivery systems disclosed herein.

The delivery system 10 and/or other components of the delivery system 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the delivery system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the delivery system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the delivery system 10 to achieve the same result.

In some embodiments, a degree of Magnetic Resonance Imaging (MRI) compatibility is imparted into the delivery system 10. For example, the delivery system 10, or portions thereof, may be made of a material that does not substantially distort the image and create substantial artifacts (e.g., gaps in the image). Certain ferromagnetic materials, for example, may not be suitable because they may create artifacts in an MRI image. The delivery system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A delivery device for an occlusive implant, the delivery device comprising:
a delivery sheath having a proximal region and a laser cut distal tip region;
a reinforcing member extending along the proximal region;
wherein the laser cut distal tip region has one or more laser cuts formed therein that form a patterned distal section, the patterned distal section being configured to shift between a delivery configuration and an expanded configuration;
a core member slidably disposed within the delivery sheath; and
an occlusive implant releasably coupled to the core member.

2. The delivery device of claim 1, wherein at least some of the one or more laser cuts go completely through a wall surface of the laser cut distal tip region.

3. The delivery device of claim 1, wherein at least some of the one or more laser cuts go only partially through a wall surface of the laser cut distal tip region.

4. The delivery device of claim 1, wherein the one or more laser cuts all extend the same distance proximally along the laser cut distal tip region.

5. The delivery device of claim 1, wherein at least some of the one or more laser cuts extend a different distance proximally along the laser cut distal tip region.

6. The delivery device of claim 1, wherein the patterned distal section includes a plurality of rounded projections.

7. The delivery device of claim 1, wherein the patterned distal section includes a plurality of pointed projections.

8. The delivery device of claim 1, wherein the patterned distal section includes axially-extending grooves.

9. The delivery device of claim 1, wherein the patterned distal section includes circumferentially-extending grooves.

10. The delivery device of claim 1, wherein the patterned distal section includes overlapping grooves.

11. A delivery device for an occlusive implant, the delivery device comprising:
a delivery sheath having a proximal region and a distal tip region;
wherein the distal tip region includes a patterned distal section;
wherein the patterned distal section is configured to allow the distal tip region to shift between a delivery configuration and an expanded configuration; and
a proximal reinforcing member disposed along the proximal region.

12. The delivery device of claim 11, wherein the patterned distal section includes laser cuts that go completely through a wall surface of the distal tip region.

13. The delivery device of claim 11, wherein the patterned distal section includes laser cuts that go only partially through a wall surface of the distal tip region.

14. The delivery device of claim 11, wherein the patterned distal section includes laser cuts that all extend the same distance proximally along the distal tip region.

15. The delivery device of claim 11, wherein the patterned distal section includes laser cuts that extend a differing distance proximally along the distal tip region.

16. The delivery device of claim 11, wherein the patterned distal section includes a plurality of rounded projections.

17. The delivery device of claim 11, wherein the patterned distal section includes a plurality of pointed projections.

18. The delivery device of claim 11, wherein the patterned distal section includes axially-extending grooves, circumferentially-extending grooves, or both.

19. The delivery device of claim 11, further comprising a core member slidably disposed within the delivery sheath and an occlusive implant releasably coupled to the core member.

20. A method for implanting an occlusive implant into a left atrial appendage, the method comprising:
advancing a delivery device to a position adjacent to the left atrial appendage, the delivery device comprising:
a delivery sheath having a proximal region and a laser cut distal tip region,
a reinforcing member extending along the proximal region,
wherein the laser cut distal tip region has one or more laser cuts formed therein that form a patterned distal section, the patterned distal section being configured to shift between a delivery configuration and an expanded configuration,
a core member slidably disposed within the delivery sheath, and
an occlusive implant releasably coupled to the core member; and
advancing the core member such that the occlusive implant advances out from the laser cut distal tip region.
